(19) United States
(12) Patent Application Publication
(10) Pub. No.: US 2009/0181911 A1
(43) Pub. Date: Jul. 16, 2009

(54) Role of gax in alzheimer neurovascular dysfunction

(76) Inventors: Benslav V. Zlokovic, Rochester, NY (US); Zhenhua Wu, Wellesley, MA (US); Rashid Deane, Rochester, NY (US)

Correspondence Address: NIXON & VANDERHYE, PC, 901 NORTH GLEBE ROAD, 11TH FLOOR, ARLINGTON, VA, 22203, US

(21) Appl. No.: 11/988,999

(22) PCT Filed: Aug. 3, 2006
(86) PCT No.: PCT/US06/30148

§ 371 Date: Mar. 18, 2008

Related U.S. Application Data
(60) Provisional application No. 60/704,903, filed on Aug. 3, 2005.

(51) Int. Cl. A61K 31/7088; C12N 15/87; C12Q 1/68; C12N 15/86; C12N 15/88

(52) U.S. Cl. 514/44; 435/455; 435/6; 435/456; 435/458

(57) ABSTRACT

Neurovascular disorder critically contributes to the development and pathogenesis of Alzheimer's disease (AD). Transcriptional profiling of human brain endothelial cells (BEC) defines a subset of age-independent genes significantly altered in AD including the homebox gene GAX whose expression controls vascular phenotype and is low in AD. By using viral-mediated GAX gene silencing and transfer, restoring GAX expression in AD BEC is angio-genic, transcriptionally suppresses the AFX1 forkhead transcription factor-mediated apoptosis, and increases the levels of a major amyloid β-peptide (Aβ) clearance receptor, the low density lipoprotein receptor-related protein 1 (LRP-1) at the blood-brain barrier. In a mouse model of Alzheimer's disease, deletion of the Gax gene results in reductions in brain capillary density and the resting cerebral blood flow, loss of angiogenic brain response to hypoxia, and an impaired Aβ brain efflux caused by reduced LRP-1 levels. The link of GAX gene to AD neurovascular dysfunction provides new mechanistic and therapeutic insights into AD.

### CROSS-REFERENCE TO RELATED APPLICATION

This is a U.S. national-stage application of Int'l Appln. No. PCT/US2006/030148 under 35 U.S.C. 371, filed Aug. 3, 2006; the entire contents of which are hereby incorporated by reference in this application. This application claims priority benefit of provisional U.S. Patent Appln. No. 60/704,903, filed Aug. 3, 2005; which is also incorporated by reference.

### STATEMENT REGARDING FEDERALLY-SPONSORED RESEARCH

The U.S. Government has certain rights in this invention as provided for by the terms of NIH-R37-AG023084 awarded by the Department of Health and Human Services.

### FIELD OF THE INVENTION

This invention relates to at least promotion of angiogenesis, suppression of apoptosis, or increase of low density lipoprotein receptor-related protein 1 (LRP-1) mediated clearance of amyloid P peptide in neurovascular cells.

### BACKGROUND OF THE INVENTION

Alzheimer's disease (AD) is the major cause of dementia in the elderly population. Since the first description of neuronal and vascular lesions in this heterogenous disorder by Alzheimer¹, there has been little understanding how the two lesions relate to each other and how they contribute to a chronic neurodegenerative disease process². Recent findings on co-morbidity of cerebrovascular disorder and AD^{3,4}, the link between atherosclerosis and AD^{5,6}, cognitive impairment associated with amyloid angiopathy⁷, major brain microvascular pathology^{8,9}, insufficient angiogenesis in AD¹⁰⁻¹² and deficient clearance of Alzheimer neurotoxin, amyloid β-peptide (Aβ), across the blood-brain barrier (BBB)¹³⁻¹⁵, indicate that neurovascular dysfunction is a critical feature of AD and could have a major impact on the pathogenesis of a chronic neurodegenerative condition.

According to the neurovascular hypothesis², dysfunction of the neurovascular unit suggests manifold pathogenic cascades for AD including: cerebrovascular flow dysregulation and hypoperfusion^{16,17}, aberrant angiogenesis and vascular remodeling¹⁰⁻¹², and faulty clearance of Aβ¹³⁻¹⁵ which all could initiate neurovascular uncoupling, vessel regression, and neurovascular inflammation, resulting in a chemical demise of the neuronal microenvironment and ultimately, synaptic and neuronal dysfunction, injury and loss. Here, we show the transcriptome profiles of human brain endothelial cells (BEC) indicate that a small subset of age-independent genes is altered in AD neurovasculature, including the homeodomain-transcription factor GAX (growth arrest-specific homeobox)¹⁸. GAX expression in the adult is restricted to the cardiovascular system and has multiple effects on the vascular phenotype¹⁹, but is low in AD neurovasculature. Restoring GAX expression in AD BEC was shown to stimulate angiogenesis, suppress AFX1 forkhead transcription factor-mediated apoptosis²⁰, and increase the levels of a major Aβ clearance receptor at the BBB, the low density lipoprotein receptor-related protein 1 (LRP)^{13,14} associated with transcriptional upregulation of its receptor associated protein (RAP)²¹. Furthermore, partial deletion of the Gax gene in mice²² results in reductions in brain capillary density and the resting cerebral blood flow, loss of brain angiogenic response to hypoxia in vivo, and a deficient Aβ clearance from brain due to reduced LRP-1 levels at the BBB associated with low expression of RAP.

### SUMMARY OF THE INVENTION

It is an object of the invention to provide an understanding of the role of GAX in neurovascular dysfunction and Alzheimer's disease.

In one embodiment, at least promotion of angiogenesis, suppression of apoptosis, increase of low density lipoprotein receptor-related protein 1 (LRP-1) mediated clearance of amyloid β peptide (Aβ), or any combination thereof is provided by a method comprising: (a) inserting a nucleic acid comprised of a GAX gene into one or more neurovascular cells and (b) expressing GAX in said neurovascular cells from said nucleic acid which is effective at least to promote angiogenesis, to suppress apoptosis, to increase LRP-1 mediated clearance of Aβ, or any combination thereof.

Further aspects of the invention will be apparent to a person skilled in the art from the following detailed description and claims, and generalizations thereto.

### BRIEF DESCRIPTION OF THE DRAWINGS

FIG. 1 shows gene expression and functional data for AD neurovascular cells. a, Relative changes in gene ontology categories in AD vs. age-matched control BEC. b, Confirmation of the microarray results for selected genes in AD vs. age-matched controls by QPCR of BEC isolated from tissue by laser capture microdissection (LCM), QPCR of cultured BEC, and immunostaining of BEC in brain tissue in situ. c, Total cortical capillary length in AD and controls used for BEC isolation (a-b) vs. clinical dementia rating (CDR) scores. d, AD BEC-mediated capillary tube formation in vitro in response to VEGF/FGF-2. e, TUNEL-positive AD BEC at 4 hr of VEGF/FGF-2 stimulation; bar 15 rpm. f, AD BEC apoptosis at 4 and 24 hr of brain capillary morphogenesis in 3-D gels. g, Brain capillary tube formation by age-matched control BEC in 3-D gels; bar 7 μm. Mean±SD, from six AD, six age-matched, and five young controls.

FIG. 2 shows that the homeobox GAX gene determines AD-like phenoltype in neurovascular cells. a-d, GAX homeoprotein (a), brain capillary tube formation in response to VEGF (b), survival of cells after VEGF stimulation (c) and expression of the forkhead transcription factor AFX1 and Bcl-X_{L} (d) in human BEC transduced with Ad.shGAX. e-f, QPCR for AFX1 mRNA in control BEC transduced with Ad.shGAX and Ad.shGFP (e) and in AD BEC transduced with human GAX (Ad.hGAX) or Ad. GFP (f). g, Brain capillary tube formation in AD BEC transduced with human Ad.hGAX or Ad.GFP. h, GAX homeoprotein, AFX1 and Bax expression in AD BEC transduced with either Ad.hGAX or Ad.GFP. Mean±SE, n=3-5. MOI, multiplicity of infection.

FIG. 3 shows that deletion of the Gax gene results in cerebrovascular incompetence in mice. a-b, Expression of Gax homeoprotein (a) and cortical cerebral blood flow (CBF) (b) in Gax^{+/-} mice. c, Total brain capillary length and brain angiogenic response to hypoxia in Gax^{+/-} and Gax^{+/+} mice. d, Brain levels of VEGF and brain capillary levels of Gax, AFX1 and Bcl-X_{L} in Gax^{+/-} and Gax^{+/+} mice subjected to hypoxia. e, In vitro angiogenesis mediated by BEC derived from Gax^{+/-} and Gax^{+/+} mice. f-g, Brain capillary levels of Gax homeoprotein in APPsw^{+/-} (Tg2576) mice and littermate controls at 18-20-month of age determined by Western blot analysis (f) and double immunostaining in tissue for CD31 (BEC marker) and Gax (g). Mean±SE, n=3-5.

FIG. 4 shows that Gax^{+/-} mice exhibit impaired Aβ clearance and LRP-1 downregulation. a-d, Aβ40 brain retention (a) and impaired clearance across the BBB (b) are associated with reduced LRP-1 levels in brain capillaries in Gax^{+/-} mice compared to Gax^{+/+} mice as determined by Western blot analysis (c) and double immunostaining (d) for LRP-1 and CD31 (endothelial marker). e, Brain capillary levels of the receptor associated protein (RAP) determined by Western blot analysis. Mean±SE, n=3-5.

FIG. 5 shows GAX-mediated regulation of LRP-1 in primary human BEC. a-b, Mature (a) and immature (b) LRP-1 levels in human BEC transduced with Ad.shGAX or Ad.shGFP. c, ³⁵S-methionine pulse-chase study for LRP-1 in BEC transduced with Ad.shGAX and Ad.shGFP. d, Effect of the proteasome inhibitor M132 on LRP-1 levels in a pulse chase experiment as in (c). e-f, The levels of RAP protein (e) and RAP mRNA (f) determined by Western blot and QPCR analyses in BEC transduced with Ad.shGAX and Ad.shGFP. g-h, Mature (g) and immature (h) LRP-1 levels in AD BEC transduced with Ad.hGAX or Ad.GFP. Mean±SE, n=3-5.

FIG. 6 shows TUNEL-positive and AFX1-positive microvessels in AD and age-matched control brains. a, TUNEL-positive brain microvessels in AD (double staining for TUNEL and collagen); bar=50 μm. b, AFX1-positive brain microvessels in AD (double immunostaining for AFX1 and CD31). c, TUNEL-positive brain microvessels are AFX1-positive (double immunostaining for TUNEL and AFX1); bar=100 μm. Mean±SE, n=5.

FIG. 7 shows vascular reactivity in Gax^{+/+} and Gax^{+/-} mice. Cumulative dose-response curves for contraction to phenylephrine a, and relaxation to acetylcholine b, of arotic rings isolated from Gax^{+/+} (open circle) and Gax^{+/-} (filled circle) mice. Values are mean±SE, n=3 mice for each group.

FIG. 8 shows brain capillary length and LRP-1 levels in Ahr^{-/-} mice. a, Total brain capillary length in AhR^{-/-} and AhR^{+/+} mice; brain microvessles are visualized with CD31 immunostaining. b, Brain capillary LRP-1 levels in AhR^{-/-} and AhR^{+/+} mice determined by Western blot analysis. Mean±SE, n=3.

FIG. 9 shows internalization of α2M in BEC with suppressed GAX expression. a, Internalization of [¹²⁵I]α2M* by human brain endothelial cells transduced with Ad.shGAX vs. Ad.shGFP. b, The half-life (t_{1/2}) for rapid LRP-1 endocytosis in human BEC transduced with Ad.shGAX vs. Ad.shGFP determined with [¹²⁵I]α2M* internalization as described¹⁴. Mean±SE, n=3.

FIG. 10 shows proteasomal proteolytic activity, transferring receptors levels, and RAP levels in human BEC. a, Proteasomal proteolytic activity in human BEC transduced with Ad.shGAX vs. Ad.shGFP. Cells were harvested 3 days after transduction, and the activity determined in cell lysates by using a proteasome assay kit (Calbiochem). Results were expressed as the percentage of Ad.shGFP control. b, Transferrin receptor (TR) levels in primary human BEC transduced with Ad.shGAX compared to cells transduced with Ad.shGFP. c, Down regulation of RAP in primary AD BEC compared to age-matched control BEC. Mean±SE, n=3.

FIG. 11 shows expression of MEF2, ankyrin G, plectin 1, and TINUR in human BEC. a, Down regulation of MEF2 in AD brain capillaries. b, Down regulation of ankyrin G and plectin 1 and primary human BEC transduced with Ad.shGAX c, Suppression of TINUR in primary human BEC transfected with plasmid encoding short hairpin interfering RNA for TINUR (shTINUR). No significant changes were observed in GAX levels in BEC transfected with shTINUR plasmid vs. control plasmid. Mean±SE, n=3.

FIG. 12 shows that Aβ does not affect GAX expression in human BEC. GAX levels in primary human BEC treated for 24 h with aggregated Aβ42 (a, agg-Aβ42), and Aβ42 oligomers (b, oligo-Aβ42) were determined by Western blot analysis. Mean±SE, n=3.

### DESCRIPTION OF SPECIFIC EMBODIMENTS OF THE INVENTION

Methods for treating Alzheimer's disease (preventive and/or therapeutic) and use of an effective amount of a nucleic acid comprised of a GAX gene methods for manufacture of a pharmaceutical composition are provided. The amount and extent of treatment administered to a cell, tissue, or subject (any animal or human) in need of therapy or prophylaxis is effective in treating the affected cell, tissue, or subject. One or more properties/functions of neurovascular cells, vascular endothelium, and endothelial cells thereof, or the number/severity of symptoms of affected subjects, may be improved, reduced, normalized, ameliorated, or otherwise treated. GAX expression is directed by choice of transcriptional regulatory region, replication of nucleic acid, and delivery by a carrier of nucleic acid. A pharmaceutical composition comprised of an effective amount of nucleic acid comprised of a GAX gene and a physiologically-acceptable vehicle, which is packaged in an aseptic container, is also provided.

Such methods may be used alone or in combination with other known methods. Instructions for performing these methods, reference values, and controls (i.e., positive/negative) may also be used. Mammals (e.g., humans and rodent or primate models of disease) may be treated. Thus, both veterinary and medical methods are contemplated.

Preparations of endothelial cells, isolated endothelium, neurovascular cells, and in vitro cell cultures are provided from brain (e.g., microvasculature) or other organs (e.g., skin) of subjects at risk for Alzheimer's disease, affected by the disease, or not. In particular, tissues like endothelium, smooth muscle, blood vessels and capillaries of the brain, temporal and leptomeningeal arteries, or any other tissues representative of vascular endothelium can be examined for GAX expression. Blood and bone marrow cells might also be used. They can be obtained as biopsy or autopsy material; cells of interest may be isolated therefrom and then cultured. Also provided are extracts of cells; at least partially purified DNA, RNA, and protein therefrom; and methods for their isolation. These reagents can be used to establish detection limits for assays, absolute amounts of gene expression that are indicative of disease or not, ratios of gene expression that are indicative of disease or not, and the significance of differences in such values. These values for positive and/or negative controls can be measured at the time of assay, before an assay, after an assay, or any combination thereof.

Nucleotide sequences representative of the GAX gene whose expression is decreased in Alzheimer's disease may be used to identify, isolate, or detect complementary nucleotide sequences by binding assays. Similarly, one or more amino acid sequences representative of GAX which are decreased in Alzheimer's disease may be used to identify, isolate, or detect interacting proteins by binding assays. Optionally, bound complexes including interacting proteins may be identified, isolated, or detected indirectly though a specific binding molecule (e.g., antibody) for GAX.

The abundance of GAX transcript or polypeptide can be measured by techniques such as in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), radioimmunoassay (RIA), fluorescent or histochemical staining, microscopy and digital image analysis, and fluorescence activated cell analysis or sorting (FACS).

An expression vector is a recombinant polynucleotide that is in chemical form either a deoxyribonucleic acid (DNA) and/or a ribonucleic acid (RNA). The physical form of the expression vector may also vary in strandedness (e.g., single-stranded or double-stranded) and topology (e.g., linear or circular). The expression vector is preferably a double-stranded deoxyribonucleic acid (dsDNA) or is converted into a dsDNA after introduction into a cell (e.g., insertion of a retrovirus into a host genome as a provirus). The expression vector may include one or more regions from a mammalian gene expressed in the microvasculature, especially endothelial cells (e.g., angiopoietin receptors Tie-1 or Tie-2, endoglin, endothelin-1 ET1, intercellular adhesion molecule ICAM-2, vascular endothelial growth factor receptors FLT-1 or FLK-1, and vascular endothelial growth factor VEGF), or a virus (e.g., adenovirus, adeno-associated virus, cytomegalovirus, herpes simplex virus, Moloney leukemia virus, mouse mammary tumor virus, Rous sarcoma virus, SV40 virus), as well as regions suitable for gene manipulation (e.g., selectable marker, linker with multiple recognition sites for restriction endonucleases, promoter for in vitro transcription, primer annealing sites for in vitro replication). The expression vector may be associated with proteins and other nucleic acids in a carrier (e.g., packaged in a viral particle or encapsulated in a liposome).

The expression vector further comprises one or more regulatory regions for gene expression (e.g., promoter, enhancer, silencer, splice donor and acceptor sites, polyadenylation signal, cellular localization sequence). Transcription from a drug-inducible regulatory region can be activated or silenced by tetracycline or dimerized macrolides. The expression vector may be further comprised of one or more splice donor and acceptor sites within an expressed region; a Kozak consensus sequence upstream of an expressed region for initiation of translation; downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include a mRNA degradation signal if it is desired to express one or more of the downstream regions only under the inducing condition. An origin of replication may be included that allows replication of the expression vector integrated in the host genome or as an autonomously replicating episome. Centromere and telomere sequences can also be included for the purposes of chromosomal segregation and protecting chromosomal ends from shortening, respectively. Random or targeted integration into the host genome is more likely to ensure maintenance of the expression vector but episomes could be maintained by selective pressure or, alternatively, may be preferred for those applications in which the expression vector is present only transiently.

An expressed region may be derived from a gene encoding GAX in operative linkage with a transcriptional regulatory region (e.g., constitutive, regulated, drug-inducible, endothelial-specific, and/or viral promoter and an optional enhancer). The expressed region may encode a translational fusion. Open reading frames of regions encoding a polypeptide and at least one heterologous domain may be ligated in register. If a reporter or selectable marker is used as the heterologous domain, then expression of the fusion protein may be readily assayed or localized.

Gene activation may be achieved by inducing an expression vector that contains a downstream region related to a GAX gene or unrelated to the GAX gene that acts to relieve suppression of gene activation (e.g., MEF2). Alternatively, the downstream expressed region may direct homologous recombination into a locus in the genome and thereby replace an endogenous transcriptional regulatory region of the gene with an expression cassette. In particular, LRP-1 expression (and transport of Aβ across the blood-brain barrier) can be induced by introduction of an exogenous GAX gene or activating an endogenous GAX gene.

An expression vector may be introduced into a host mammalian cell or non-human mammal by a transfection or transgenesis technique using, for example, chemicals (e.g., calcium phosphate, DEAE-dextran, lipids, polymers), biolistics, electroporation, naked DNA technology, microinjection, or viral infection. The introduced expression vector may integrate into the host genome of the mammalian cell or non-human mammal. Many neutral and charged lipids, sterols, and other phospholipids to make lipid carrier vehicles are known. For example, neutral lipids are dioleoyl phosphatidylcholine (DOPC) and dioleoyl phosphatidyl ethanolamine (DOPE); an anionic lipid is dioleoyl phosphatidyl serine (DOPS); cationic lipids are dioleoyl trimethyl ammonium propane (DOTAP), dioctadecyldiamidoglycyl spermine (DOGS), dioleoyltrimethyl ammonium (DOTMA), and 1,3-di-oleoyloxy-2-(6-carboxy-spermyl)-propylamide tetraacetate (DOSPER). Dipalmitoyl phosphatidylcholine (DPPC) can be incorporated to improve the efficacy and/or stability of delivery. FUGENE 6, LIPOFECTAMINE, LIPOFECTIN, DMRIE-C, TRANSFECTAM, CELLFECTIN, PFX-1, PFX-2, PFX-3, PFX-4, PFX-5, PFX-6, PFX-7, PFX-8, TRANSFAST, TFX-10, TFX-20, TFX-50, and LIPOTAXI lipids are proprietary formulations. The polymer may be polyethylene glycol (PEG) or polyethylenimine (PEI); alternatively, polymeric materials can be formed into nanospheres or microspheres. Naked DNA technology delivers the expression vector in plasmid form to a cell, where the plasmid may or may not become integrated into the host genome, without using chemical transfecting agents (e.g., lipids, polymers) to condense the expression vector prior to introduction into the cell.

Thus, a mammalian cell may be transfected with an expression vector; also provided are transgenic nonhuman mammals. In the previously discussed alternative, a homologous region from a gene can be used to direct integration to a particular genetic locus in the host genome and thereby regulate expression of the gene at that locus. Polypeptide may be produced in vitro by culturing transfected cells; in vivo by transgenesis; or ex vivo by introducing the expression vector into allogeneic, autologous, histocompatible, or xenogeneic cells and then transplanting the transfected cells into a host organism. Special harvesting and culturing protocols will be needed for transfection and subsequent transplantation of host stem cells into a host mammal. Immunosuppression of the host mammal post-transplant or encapsulation of the host cells may be necessary to prevent rejection.

The expression vector may be used to replace the function of a gene that is down regulated or totally defective or supplement function of a partially defective gene. Thus, the cognate gene of the host may be neomorphic, hypomorphic, hypermorphic, or normal. Replacement or supplementation of function can be accomplished by the methods discussed above, and transfected mammalian cells or transgenic nonhuman mammals may be selected for high expression (e.g., assessing amount of transcribed or translated product, or physiological function of either product) of the downstream region.

Nucleic acids may be used to formulate a pharmaceutical composition with one or more of the utilities disclosed herein. Use of a physiologically acceptable vehicle and compositions which further comprise carriers for delivering a nucleic acid to a subject are known in the art. Addition of such vehicles and carriers to the composition is well within the level of skill in this art. Compositions may be administered in vitro to cells in culture, in vivo to cells in the body, or ex vivo to cells outside of the subject that may later be returned to the body of the same subject or another. Such cells may be diaggregated or provided as solid tissue.

Pharmaceutical compositions may be administered as a formulation adapted for passage through the blood-brain barrier or direct contact with the endothelium. Alternatively, pharmaceutical compositions may be added to the culture medium. In addition to the nucleic acid, such compositions may contain a physiologically-acceptable vehicle and other ingredients known to facilitate administration, condense the nucleic acid, enhance uptake, or any combination thereof (e.g., saline, dimethyl sulfoxide, lipid, polymer, affinity-based cell specific-targeting systems). The composition may be incorporated in a gel, sponge, or other permeable matrix (e.g., formed as pellets or a disk) and placed in proximity to the endothelium for sustained, local release. The composition may be administered in a single dose or in multiple doses which are administered at different times.

Pharmaceutical compositions may be administered by any known route. By way of example, the composition may be administered by a topical (e.g., epidermal, mucosal, or pulmonary) or other localized or systemic route (e.g., enteral and parenteral). The term “parenteral” includes subcutaneous, intradermal, intramuscular, intravenous, intra-arterial, intrathecal, and other injection or infusion techniques, without limitation.

Suitable choices in amounts and timing of doses, formulation, and routes of administration can be made with the goals of achieving a favorable response in the subject with Alzheimer's disease or at risk thereof (i.e., efficacy), and avoiding undue toxicity or other harm thereto (i.e., safety). Therefore, “effective” refers to such choices that involve routine manipulation of conditions to achieve a desired effect.

A bolus administered once a day is a convenient dosing schedule. Alternatively, the effective daily dose may be divided into multiple doses for administration, for example, two to twelve doses per day. Dosage levels of active ingredients in a pharmaceutical composition can also be varied so as to achieve a transient or sustained concentration of the nucleic acid in a subject, especially in and around vascular endothelium of the brain (neurovascular cells), and to result in the desired therapeutic response or protection. But it is also within the skill of the art to start doses at levels lower than required to achieve the desired effect and to gradually increase the dosage until the desired effect is achieved.

The amount of nucleic acid administered is dependent upon factors known to a person skilled in the art such as its bioactivity and bioavailability (e.g., half-life in the body, stability, and metabolism); its chemical properties (e.g., molecular weight, hydrophobicity, and solubility); route and scheduling of administration; and the like. It will also be understood that the specific dose level to be achieved for any particular subject may depend on a variety of factors, including age, gender, health, medical history, weight, combination with one or more other drugs, and severity of disease.

The term “treatment” of Alzheimer's disease refers to, inter alia, reducing or alleviating one or more symptoms in a subject, preventing one or more symptoms from worsening or progressing, promoting recovery or improving prognosis, and/or preventing disease in a subject who is free therefrom as well as slowing or reducing progression of existing disease. For a given subject, improvement in a symptom, its worsening, regression, or progression may be determined by an objective or subjective measure. Efficacy of treatment may be measured as an improvement in morbidity or mortality (e.g., lengthening of survival curve for a selected population). Prophylactic methods (e.g., preventing or reducing the incidence of relapse) are also considered treatment. Treatment may also involve combination with other existing modes of treatment (e.g., ARICEPT or donepezil, EXELON or rivastigmine, anti-amyloid vaccine, mental exercise or stimulation). Thus, combination treatment with one or more other drugs and one or more other medical procedures may be practiced.

The amount which is administered to a subject is preferably an amount that does not induce toxic effects which outweigh the advantages which result from its administration. Further objectives are to reduce in number, diminish in severity, and/or otherwise relieve suffering from the symptoms of the disease as compared to recognized standards of care.

Production of nucleic acids according to present regulations will be regulated for good laboratory practices (GLP) and good manufacturing practices (GMP) by governmental agencies (e.g., U.S. Food and Drug Administration). This requires accurate and complete recordkeeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

The following examples are merely illustrative of the invention, and are not intended to restrict or otherwise limit its practice.

### EXAMPLES

### Transcriptional Profiling of Human BEC

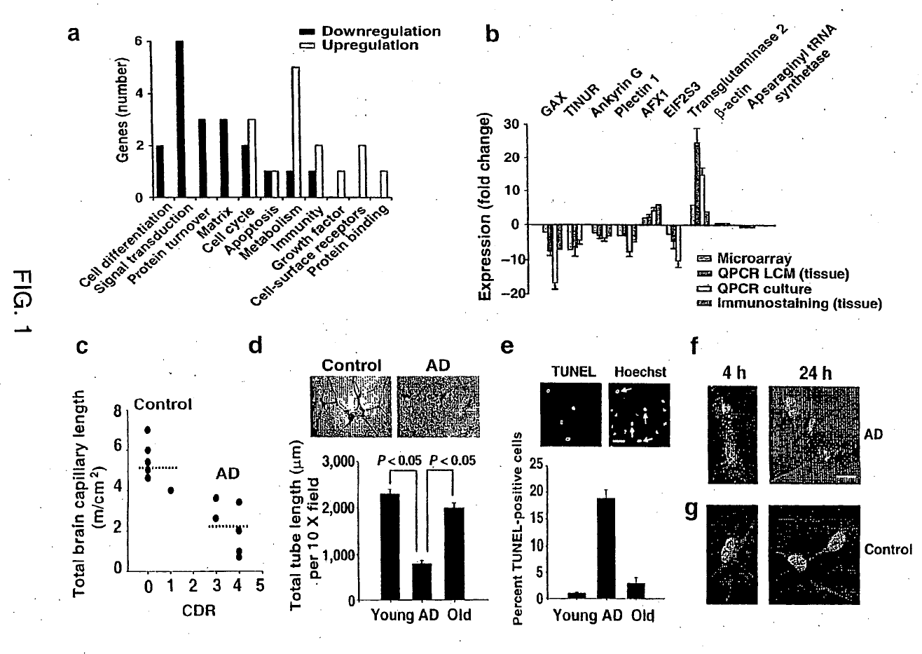

To identify novel targets in AD neurovasculature, transcriptional profiling of human BEC derived from rapid brain autopsies from the frontal pole from 36 individuals was performed. First, six AD patients with severe pathology (Braak—V-VI²³, CERAD (Consortium to Establish a Registry for Alzheimer's Disease protocol)—frequent or moderate²⁴, clinical dementia rating (CDR) score—4, age—70 yrs); six neurologically normal non-demented age-matched controls with no or sparse pathology (Braak—0 or 0-I, CERAD—negative or sparse, dementia score—0, age—70 yrs); and five young controls with no pathology (age—24 yrs) were compared. There were no differences in gender, cause of death and incidence in the vascular risk factors between AD and age-matched controls (Table 1). Comparison of the transcriptome profiles (Affymetrix U95A) by the Bayesian t-test²⁵ indicated a small subset of 34 genes, or 0.27% of approximately 12,600 genes studied, were significantly (P<0.05) altered in AD (2-fold or more, FIG. 1a; Table 2), but not between young and age-matched controls. The genes with a role in cell differentiation, signal transduction, protein turnover and matrix were downregulated, whereas genes with a role in the cell cycle, apoptosis, metabolism, immunity and cell surface receptors were either downregulated or upregulated (FIG. 1a).

A subset of functionally important genes was next validated by quantitative polymerase chain reaction (QPCR) of BEC isolated from brain tissue by laser capture microdissection and of BEC in culture, and by immunostaining of microvascular endothelium in brain tissue in situ (FIG. 1b). The validation analysis confirmed the results of the microarray analysis by demonstrating low levels of the homeobox GAX gene¹⁸; downregulation of TINUR/NURR1, a transcription factor which regulates angiogenesis²⁶; downregulation of ankyrin G and plectin 1 implicated in angiogenic responses¹²; upregulation of the proapoptotic forkhead transcription factor AFX1²⁰; upregulation of tissue transglutaminase 2 implicated in protein cross-linking²⁷; and reduced expression of the key translation initiation factor 2 (EIF2S3) required for protein synthesis²⁸.

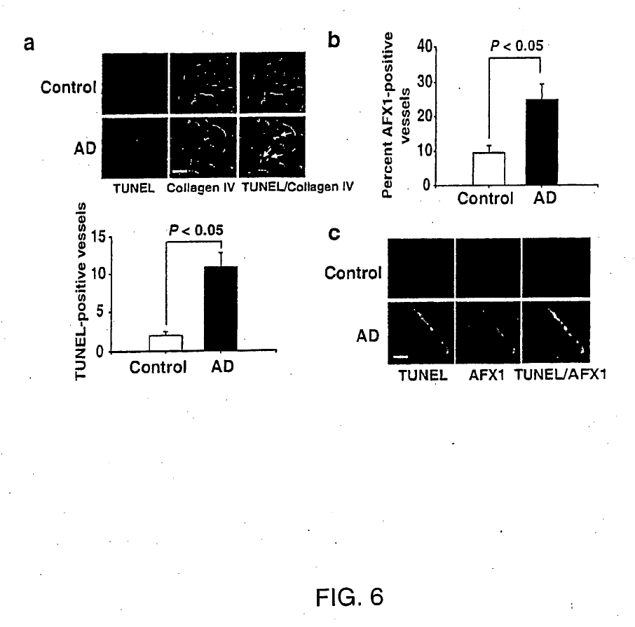

Total brain capillary length in AD cortical tissue used for the BEC study (FIGS. 1a-1b) was reduced by about 60% compared to age-matched controls, and was inversely related to the CDR scores for dementia (FIG. 1c) as reported⁸. In response to stimulation with VEGF/FGF-2 (vascular endothelial growth factor/fibroblast growth factor), AD BEC formed approximately 65% less capillary tubes than controls (FIG. 1d). About 20% of AD BEC were TUNEL (terminal deoxynucleotidyl transferase-mediated in situ end labeling)-positive within 4 hr of exposure to VEGF/FGF-2 in contrast to 1 to 3% in controls (FIG. 1e). DNA fragmentation (laddering) was confirmed by electrophoresis. Apoptotic changes in AD BEC (e.g., nuclear condensation, fragmentation, cellular shrinkage) were seen during the lumen formation (stage I)²⁹ (FIG. 1f, 4 h) and tube elongation (stage II)²⁹ (FIG. 1f, 24 h) in contrast to control BEC (FIG. 1g). The number of TUNEL-positive microvessels in brain tissue in situ was higher in AD than in controls, i.e., 12% vs. 2%, respectively (FIG. 6a), and brain capillaries in AD expressed substantially higher levels of the active form of caspase-3.

### GAX Regulates Human BEC-Mediated Angiogenesis

Since homeobox genes play important roles in the final transcriptional regulation of pathways mediating angiogenesis and differentiation of vascular cells¹⁹, it was hypothesized that low levels of GAX expression in AD neurovasculature (FIG. 1b) may critically influence BEC regression in situ (FIGS. 1c and 6a) and apoptosis in vitro (FIGS. 1d-1f, whereas restoring GAX expression may correct neurovascular dysfunction in AD. A second series of transcriptional profiling studies of BEC from ten AD patients with severe pathology (Braak V-VI) and nine age-matched controls with no or sparse pathology (Braak 0-I) confirmed low expression of GAX mRNA in AD.

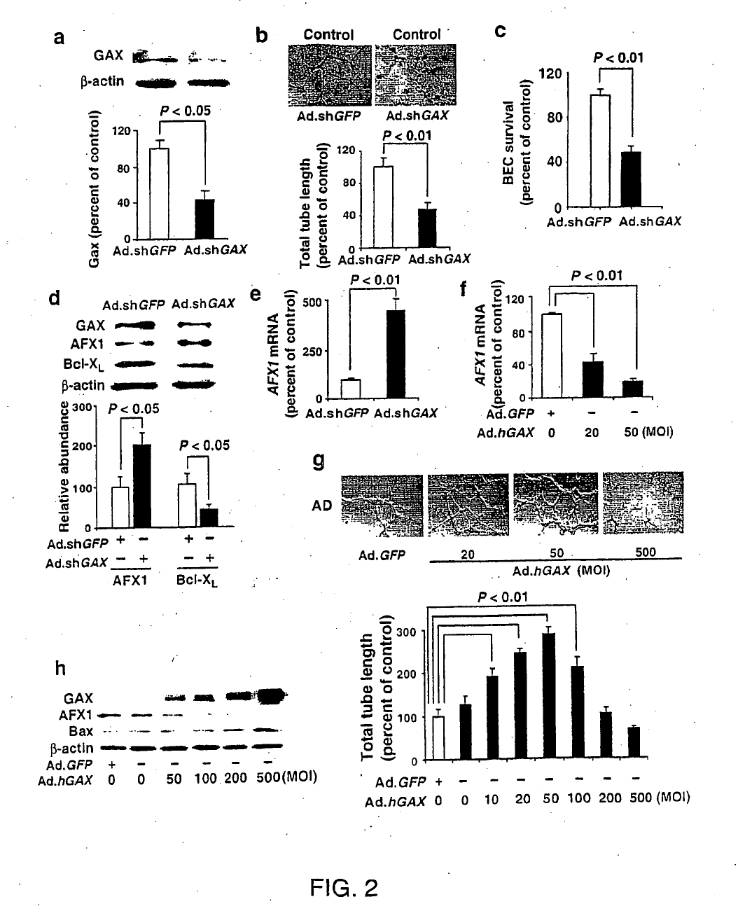

To determine the role of GAX in brain angiogenesis, human BEC were transduced with replication-incompetent adenovirus containing a short hairpin silencing double-stranded oligonucleotide construct specific for the GAX gene (Ad.shGAX). Human BEC transduced with GAX gene specific silencer, as compared to controls (Ad.shGFP, green fluorescence protein), express 40% of GAX homeoprotein (FIG. 2a), form 60% less capillary tubes (FIG. 2b) and exhibit substantially lower survival rates after VEGF stimulation (FIG. 2c).

GAX gene silencing increased by about 2-fold the levels of the AFX1 transcription factor in BEC (FIG. 2d) consistent with its increased expression in AD BEC (FIGS. 1b and 6b). AFX1 regulates apoptosis by suppressing the anti-apoptotic Bcl-X_{L} protein²⁰ which was suppressed in BEC transduced with Ad.shGAX (FIG. 2d), and in AD BEC in situ and in culture. In AD, TUNEL-positive brain microvessels were also AFX1-positive (FIG. 6c). Silencing GAX upregulated BEC AFX1 mRNA by 4.5-fold (FIG. 2e), whereas transduction of AD BEC with human GAX gene (Ad.hGAX) dose-dependently suppressed AFX1 mRNA (FIG. 2f), suggesting GAX is a transcriptional repressor of AFX1.

To confirm that restoring GAX levels may correct aberrant AD BEC-mediated angiogenesis, AD BEC were transduced with a human GAX gene (Ad.hGAX). Transfer of GAX gene at a low multiplicity of infection (MOI) increased VEGF-mediated brain capillary tube formation by 2.8-fold (FIG. 2g). In contrast, transfer of GAX gene to human umbilical vein cells (HUVEC) was antiangiogenic³⁰. Differential responses of AD BEC vs. HUVEC³⁰ to GAX gene transfer could be explained by differences in the cell types and/or by different basal levels of GAX expression in cells prior to gene transfer, i.e., extremely low in AD BEC compared to unsuppressed levels in HUVEC³⁰.

To further understand possible discrepancy between previous work^{30,31} and the current results, the dose response of AD BEC to GAX gene transfer was studied. The angiogenic effect of GAX gene transfer into AD cells followed a U-shape curve with a plateau at 50-100 MOI, whereas forced GAX expression at a higher (i.e., 500) MOI was moderately antiangiogenic (FIG. 2g). GAX gene transfer dose-dependently inhibited the AFX1 proapoptotic pathway (FIG. 2h), but at MOI≧200 also increased the levels of Bax in AD BEC (FIG. 2h), consistent with a report showing that forced GAX gene transfer induces Bax-mediated apoptosis³².

Thus, an “effective” amount of nucleic acid comprised of a GAX gene or an “effective” amount of GAX expression is empirically determined in comparison to the GAX expression conferred in AD BEC transduced with Ad.hGAX preferably at MOI less than about 200, more preferably at MOI less than about 150, or even more preferably at MOI less than about 100. MOI more than about 50 is also preferred.

### Gax Deletion Alters Brain Angiogenesis in Mice

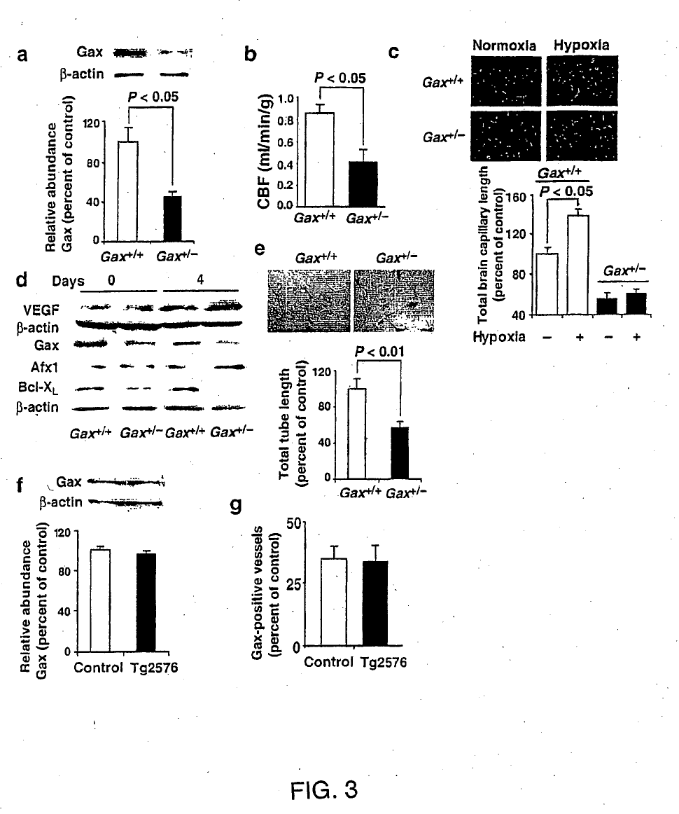

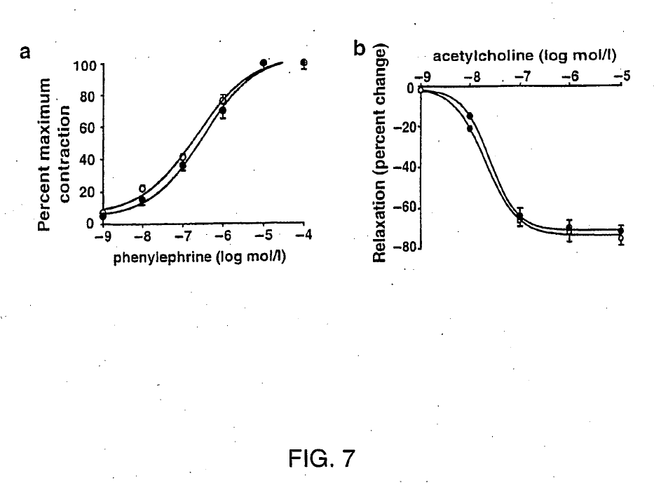

To determine whether Gax affects brain microcirculation in vivo, Gax^{+/-} mice²², which compared to Gax^{+/+} mice express <50% of brain capillary Gax homeoprotein (FIG. 3a), were studied. At 2-3 months of age, Gax^{+/-} mice had about 50% reductions in the cortical cerebral blood flow (CBF) determined with ¹⁴C-iodoantipyrine³³ (FIG. 3b; similar results were seen for other brain regions), and the total cortical capillary length (FIG. 3c; similar results were seen for other brain regions). Deletion of the Gax gene was not associated with the altered vascular reactivity, and the dose response curves of the isolated aortic rings to acetylcholine and phenylephrine were comparable to those obtained in Gax^{+/+} mice (FIG. 7).

To establish whether normal Gax expression is required for angiogenesis in vivo, the brain response to hypoxia was determined in Gax^{+/-} and Gax^{+/+} mice using an established hypoxia model in which brain angiogenesis is driven by endogenous VEGF³⁴. After three weeks of hypoxia, Gax^{+/+} mice increased brain capillary length by 38%, whereas Gax^{+/-} mice did not exhibit a significant change in brain capillary density (FIG. 3c). During hypoxia, brain levels of VEGF in Gax^{+/+} and Gax^{+/-} mice were similarly increased, but Gax brain capillary levels were substantially lower in Gax^{+/-} mice as expected, and were associated with increased levels of AFX1 and downregulation of the antiapoptotic Bcl-X_{L} (FIG. 3d) as seen in human BEC (FIG. 2d). BEC derived from Gax^{+/-} mice compared to Gax^{+/+} mice formed 55% less capillary tubes in response to VEGF (FIG. 3e).

Also studied was deposition of Aβ alters Gax expression in Alzheimer Tg2576 APPsw^{+/-} mice at 18-20 months of age when significant brain and vascular Aβ and amyloid accumulations develop^{35,36}. Brain microvascular Gax was not affected by Aβ in these Tg2576 mice (FIGS. 3f-3g). Since recent reports suggest that aggregated Aβ is antiangiogenic, and that APPsw^{+/-} mice have reduced brain capillary density^{10,11}, low levels of GAX and accumulation of Aβ may act in concert to inhibit angiogenesis in AD.

### Reduced Aβ Clearance in Gax^{+/-} Mice

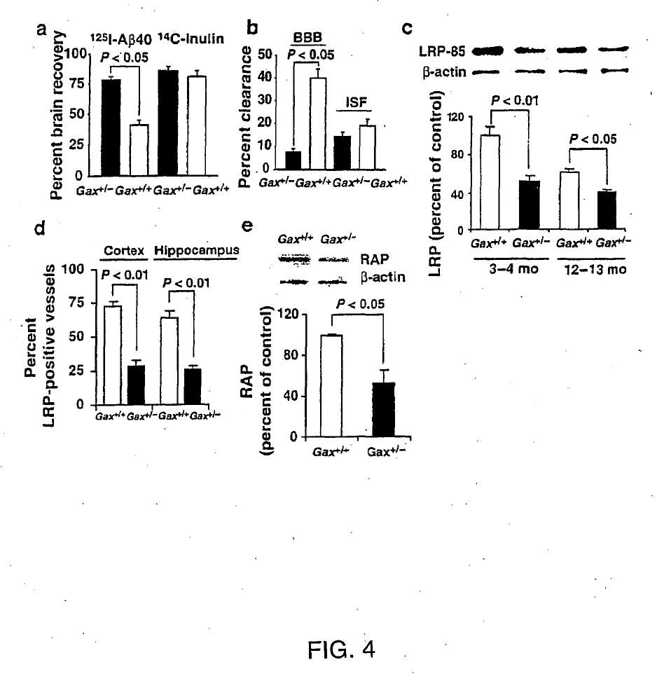

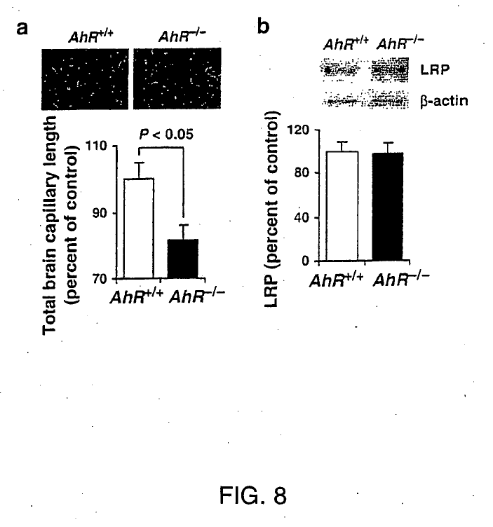

Reduced vascular competence and possibly incomplete BEC differentiation in Gax^{+/-} mice was studied to determine whether Aβ clearance from brain interstitial fluid (ISF) was affected, using methods as described^{13,14}. Gax^{+/-} mice, as compared to Gax^{+/+} mice, showed substantial Aβ40 brain retention (FIG. 4a). Moreover, rapid elimination of Aβ40 across the BBB was greatly reduced by about 80% in Gax^{+/-} mice (FIG. 4b), whereas its clearance via slow ISF bulk flow, which represents a minor component of Aβ efflux from brain¹³, was not affected (FIG. 4b). It was then asked whether brain capillary levels of LRP, the major clearance receptor for Aβ at the BBB^{13,14}, are altered in Gax^{+/-} mice. Western blot analysis indicated about 60% reduction in LRP-1 in Gax^{+/-} vs. Gax^{+/+} mice at 3-4 months of age (FIG. 4c), which has been corroborated by the immunostaining analysis of brain tissue in situ demonstrating that only about 25% of brain microvessels are LRP-positive in Gax^{+/-} mice compared to 70-75% LRP-positive vessels in controls (FIG. 4d). Further reductions in LRP-1 were found in Gax^{+/-} mice at 9-12 months of age, but an age-dependent down-regulation of LRP-1 was also seen (FIG. 4c), although the capillary density did not change with the age as reported^{13,14}. RAP, which regulates LRP-1 by controlling its trafficking and folding^{21,37}, was reduced by 50% in Gax^{+/-} mice (FIG. 4e). In contrast, brain LRP-1 levels were not altered in aryl hydrocarbon receptor (AhR^{-/-}) deficient mice which exhibit vascular abnormalities and reduced vascular density in many organs³⁸ including the brain (FIG. 8), which suggests LRP-1 downregulation may not necessarily accompany the reductions in capillary density.

### GAX Regulates LRP-1 Expression in Human BEC

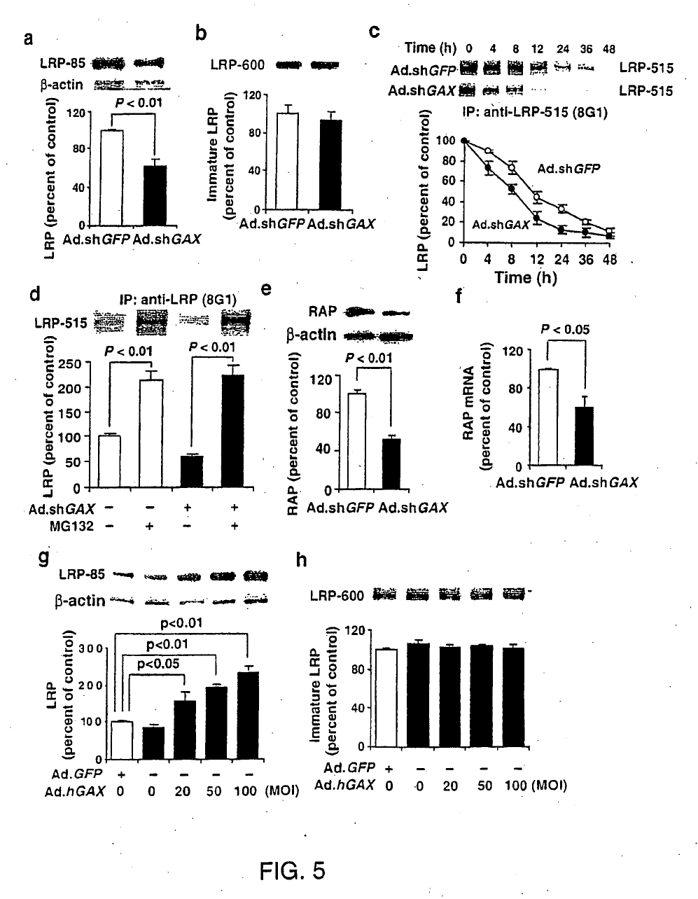

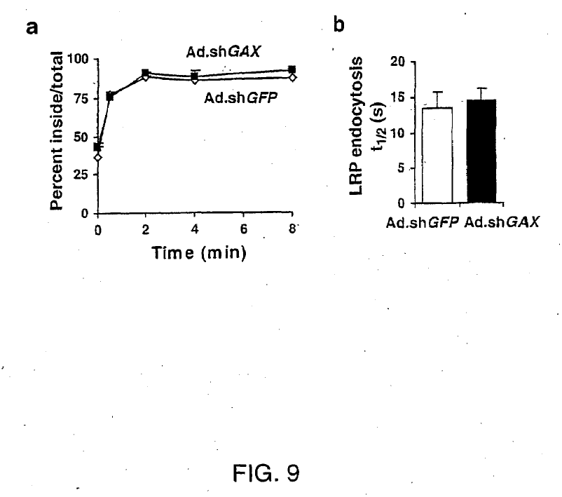

Silencing GAX gene expression substantially reduced LRP-1 expression in BEC (FIG. 5a), but did not affect LRP-1 synthesis as demonstrated by unchanged levels of immature LRP-1 (600 kDa; FIG. 5b), which reflect newly synthesized LRP-1 prior to its final subunits assembly and transport to the cell membrane^{21,37,39}. A pulse-chase experiment¹⁴ has shown that GAX gene suppression decreases the half-life of LRP-1 from 12.4 to 6.6 hr (FIG. 5c). MG132, an inhibitor of the proteasome-dependent LRP-1 degradation^{14,39}, normalized LRP-1 levels in Ad.shGAX transduced BEC (FIG. 5d). GAX did not affect LRP-1 internalization rate as indicated by unchanged endocytosis of α2-macroglobulin, a specific LRP-1 ligand³⁹, in Ad.shGAX transduced cells (FIG. 9).

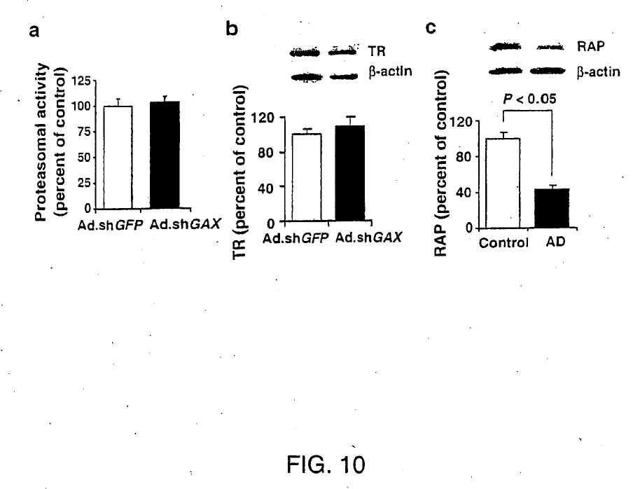

Since silencing GAX gene did not affect the proteasomal proteolytic activity (FIG. 10a) or the levels of other receptors, i.e., the transferrin receptor (FIG. 10b), a direct influence of GAX on the expression of RAP was studied. Without RAP, LRP-1 is a subject to faster turnover due to improper folding and/or abnormal aggregation²¹. Silencing GAX gene reduced RAP protein and mRNA levels by 55% and 40%, respectively (FIGS. 5e-5f). RAP was reduced by about 60% in AD BEC (FIG. 10c), consistent with a 25% decrease in RAP mRNA by the microarray analysis (p=0.11). Transduction of AD BEC with human GAX gene dose-dependently restored LRP-1 levels (FIG. 5g), but did not affect synthesis of immature LRP-1 (FIG. 5h).

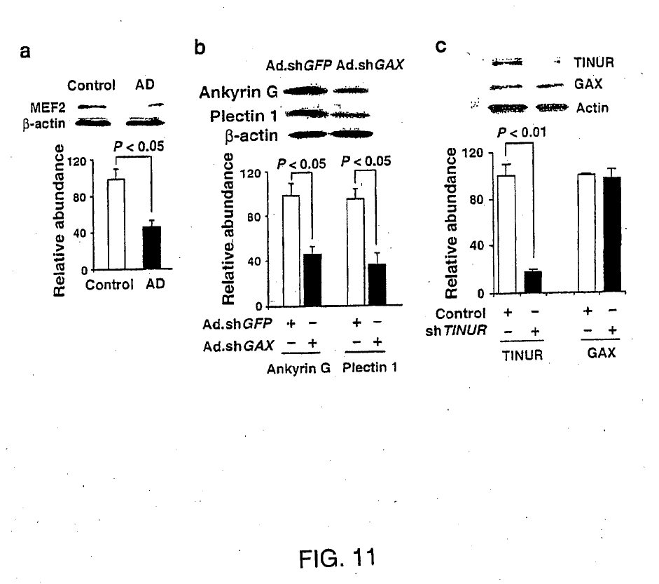

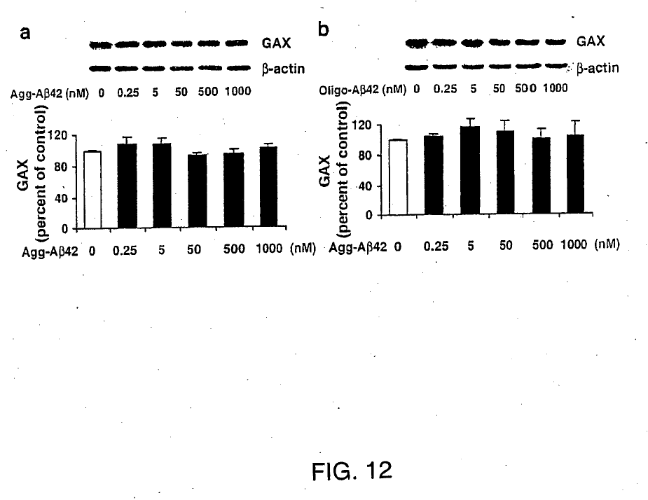

To address possible upstream events in AD BEC leading to reduced GAX expression, expression of the myocyte-specific enhancer factor-2 (MEF2) (FIG. 11a), which transactivates GAX promoter⁴⁰, was reduced by 60%. Genes with a role in angiogenesis whose expression was down regulated in AD (FIG. 1b) (e.g., ankyrin, plectin-1¹²) were downstream of GAX (FIG. 11b), as well as AFX1 (FIGS. 2e-2f), whereas silencing TINUR did not affect GAX (FIG. 11c), and vice versa. Aβ42 oligomers and/or aggregated forms did not affect GAX levels (FIG. 12).

### Discussion

Recent findings suggest that altered brain capillary-unit physiology, compromised brain microcirculation, vascular neuroinflammatory response and disruption of brain activity-mediated CBF regulation are of major importance for the pathogenesis of cognitive decline in AD^{2,18}. The present data suggest that low expression of vascularly-restricted homebox GAX gene in AD BEC mediates an aberrant angiogenesis, activates the AFX1-dependent proapoptotic pathway²⁰ and suppresses expression of the LRP-1 clearance receptor for Aβ¹³⁻¹⁵ at the BBB. Thus, GAX may control a major neurovascular disease pathway in AD.

Since low GAX levels in AD brains in situ was shown to correspond to the low, left end of the angiogenic AD BEC curve in vitro, one would expect that restoring GAX expression in AD will promote angiogenesis and vascular remodeling and inhibit the AFX1-mediated apoptosis²⁰. On the other hand, forced GAX expression in neurovasculature at high MOI may activate Bax and could be antiangiogenic likely due to Bax-mediated apoptosis³². But, extremely high levels of GAX as those achieved during forced GAX expression are seen only under the experimental conditions in vitro at the right end of the U-shape angiogenic curve, and are not found normally in healthy human brain or in a disease state.

No change in Gax expression in Tg2576 mice, which do not show neuronal death³⁶ and low GAX expression in AD patients which have neuronal loss^{23,24} were demonstrated. This indicates that neuronal loss and GAX loss are associated with each other, but whether neuronal loss in AD precedes or is secondary to the changes in GAX expression is not clear at present. With respect to the link with oxidative stress which kills neurons and may directly down regulate GAX through activation of redox-sensitive mitogen activated protein kinase⁴¹, Aβ is the chameleon of the two worlds and exhibits both pro-oxidant and antioxidant properties⁴², and therefore may not necessarily affect GAX expression. In vitro data confirmed that exposure of BEC to Aβ42 oligomers and/or aggregated forms does not suppress GAX, as found in Tg2576 mice in vivo.

There is evidence that cerebral hypoperfusion impairs neuron metabolism and compromises protein synthesis that is essential for memory formation and plasticity^{9,43}. Cerebral protein synthesis is suppressed at the CBF reductions between 30% and 50%⁴³, as seen in Gax^{+/-} mice, which may suggest that neuronal function in these mice, and perhaps in AD patients with reduced CBF, could be affected even though they do not have an outright stroke.

In conclusion, the homebox GAX gene may play an important role in neurovascular dysfunction in AD relevant to AD pathology. Its low expression in AD neurovasculature and in an animal model of the Gax gene partial deletion may lead (1) to impaired angiogenesis associated with apoptosis, vessel malformation and regression ultimately resulting in reductions in brain capillary density and CBF, as seen in AD^{2,3,8,9,16}; and (2) to a pathological BBB phenol-type with little or no Aβ clearing capability due to low levels of LRP, which may lead to Aβ accumulation¹³⁻¹⁵ as seen in AD models and AD^{2,15}. Thus, GAX could be a potential new therapeutic target for AD neurovascular disorder.

### Materials and Methods

Patients and neuropathological diagnosis. BEC were isolated from rapid brain autopsies from the frontal pole (area 9/10) from 36 individuals. AD patients and age-matched controls were evaluated clinically and followed to autopsy at the AD Research Centers at the University of Southern California and the University of Rochester Medical Center. The CDR scores in AD and control individuals were 4-3 and 0, respectively. AD cases were Braak stage V-VI²³ and CERAD²⁴ frequent to moderate; controls were Braak 0 or 0-I and CERAD negative or sparse. In the first group, BEC transcriptome profiles from six AD patients, six age-matched controls, and five young controls were compared. For clinical and neuropathological characteristics see Table 1. The incidence of vascular risk factors (e.g., hypertension, atherosclerosis, etc.), the gender ratio, age, cause of death and the post-mortem interval were comparable between AD and age-matched controls. BEC from young controls (average age 23.4 years) were isolated from rapid brain autopsies of neurologically normal young individuals with no vascular risk factors autopsied after motor vehicle accidents at the Monroe Medical Examiner Center, New York. The microarray analysis of these young control BEC did not reveal significant differences in gene expression profiles compared with BEC derived from control cortical brain tissue after epilepsy surgery from young individuals of comparable age and gender. In the second group, BEC transcriptome profiles were compared in ten AD patients with severe pathology vs. nine age-matched controls with no or sparse pathology. The gender ratio, age, cause of death, the post-mortem interval and the incidence of vascular risk factors were comparable between AD and controls.

Laser capture microdissection (LCM) of BEC. Autopsy specimen of the frontal cortex (area 9/10) were snap frozen and cut (10 μm) using the Microm HM 500M cryostate. Cryosections were fixed for 5 min in ice cold acetone and air-dried. Capillary BEC were stained with biotinylated Ulex lectin (Vector 1:10) and treated with RNA SECURE (Ambion) employing ABC-peroxidase (Vector) and DAB. Contamination free LCM⁴⁴ was done from dry, stained sections at 400× magnification by Zeiss AXIOVERT 200 inverted microscope equipped with PALM LCM system including a 337 nm laser and a robotic microscope table operated by the PALMROBO software. RNA was isolated from single cells or aggregates of 100-250 cells by the Zymo MINI RNA ISOLATION kit (Zymo Research #R1005). cDNA was made and two rounds linear amplification performed by the Ambion MESSAGE AMP aRNA kit. The quality and normal size distribution of cDNA fragments was controlled by Agilent 2100 Bioanalyzer using the NANO chip. The magnitude of aRNA amplification using the Ambion MESSAGE AMP aRNA kit was on the order of 10⁵-10⁶.

High-density oligonucleotide array hybridization. Total RNA was prepared from BEC with TRIZOL protocol (Gibco BRL). cDNA was synthesized, in vitro transcribed and hybridized to Affymetrix HG-U95A chip containing approximately 12,600 full-length cDNA from the UNIGENE cluster database. Statistical analysis was performed by the Bayesian t-test²⁵ using the following criteria: at least 2-fold ratio of the Affymetrix signal, minimal signal of 500 (expression), and P values <0.05. Data were logarithmically transformed prior to statistical analysis. For selected genes, validation of the microarray results was performed by QPCR analysis of BEC isolated from tissue by LCM, QPCR analysis of cultured BEC (see below), and by immunostaining of BEC in tissue in situ.

Quantitative RT-PCR (QPCR). mRNA quantification was performed using TAQMAN™ chemistry with fluorescently tagged oligonucleotide probes⁴⁵. Fluorescent intensity was detected by the Perkin-Elmer Applied Biosystem Sequence Detector 7700. Data were analyzed using Perkin-Elmer Sequence Detector Software version 1.6.3. Comparative analysis was performed using the delta-delta Ct approach as described by Applied Biosystems. The same cDNA was used for microarray hybridization and QPCR analysis.

Immunostaining of BEC in human tissue. Immunocytochemical analysis of selected proteins on brain microvessels in tissue was performed on paraffin sections (6 μm) of the frontal cortex (area 9/10) adjacent to the site of BEC LCM isolation. Antigen retrieval was performed by treating tissue sections with BD Retrievagen B (BD PharMingen, San Diego, Calif.). Image analysis was performed using Olympus AX70 microscope equipped with the SPOT digital camera. Ten randomly selected fields in each region from ten sections from Brodman A9/10 areas were analyzed. Monoclonal mouse antibody to human collagen IV (1:25, 75 mg/L; DAKO, A/S, Denmark) or polyclonal rabbit antibody to human Von Willebrand Factor (1:200, 5.7 mg/ml; DAKO, A/S, Denmark) were used to label microvessels, and fluorescein goat antibody to mouse IgG (1:150, 2 mg/ml; Molecular Probe, Eugene, Oreg.) was used as a secondary antibody. GAX was detected with polyclonal rabbit antibody against rat Gax which crossreacts with human GAX (1:200, gift from Dr Kenneth Walsh Boston University⁴⁹) and secondary rhodamine goat antibody to rabbit IgG (1:150, 2 mg/ml); AFX1 with polyclonal rabbit antibody to human AFX1 (1:1000, 0.1 mg/ml; Sigma, St. Louis, Mo.) and rhodamine goat antibody to rabbit IgG (1:150, 2 mg/ml); ANK3 with monoclonal mouse antibody to human ankyrin G (1:100, 0.2 mg/ml; Santa Cruz Biotechnology, Santa Cruz, Calif.) and rhodamine goat antibody to mouse IgG (1:150, 2 mg/ml); PLEC1 with polyclonal goat antibody to human plectin 1 (1:100, 0.2 mg/ml; Santa Cruz Biotechnology) and rhodamine goat antibody to mouse IgG (1:150, 2 mg/ml); TGM2 with polyclonal rabbit antibody to human transglutaminase 2 (1:100,1 mg/ml; Calbiochem, San Diego, Calif.) and rhodamine goat antibody to rabbit IgG (1:150, 2 mg/ml).

Brain capillary length in human tissue. The paraffin-embedded coronal sections (6 μm thickness; adjacent to the BEC LCM isolation site) of each individual were cut and sampled in a systematic uniform random manner for each AD patient or control subject. Sections were immunostained for human Von Willebrand Factor in order to label vessels (10 sections per subject). Total brain capillary length was determined using IMAGEPRO PLUS software, similar as reported⁸.

Human BEC cultures. Primary human BEC were isolated from the frontal pole (area 9/10) adjacent to the site of BEC LCM isolation and immunostaining. BEC were sorted by flow activated cell sorting with Dil-Ac-LDL and characterized as reported⁴⁶. Cells were cultured (10% fetal calf serum, 10% Nuserum, endothelial cell growth factors, nonessential amino acids, vitamins and penicillin/streptomycin in RPMI 1640) in 5% CO2 at 37° C. BEC were >98% positive for endothelial markers Factor VIII and CD105, and negative for CD11b (monocyte/microglia), glial fibrillar acidic protein (astrocytes) and α-actin (vascular smooth muscle)⁴⁸. Early passage (P2-P4) cultures were used throughout the study.

GAX silencing by RNA interference, The BLOCK-iT Adenoviral RNAi expression system (Invitrogen)⁴⁷ was used. A short hairpin silencing double-stranded oligonucleotide construct for the Gax gene was designed according to the MPI algorithm (see Tuschl Lab website). A selected sequence GGAAGGAAATTAC AAGTCAGA (SEQ ID NO:1) was cloned into the BLOCK iT U6 RNAi expression entry vector. The siRNA expression cassette was recombined into the adenoviral destination vector pAD/BLOCK-iT-DEST, which was transduced into HEK 293A cells for production of recombinant replication incompetent adenovirus. After virus particle purification by VIRAKIT ADENOMINI-4 (Virapur) and determination of virus titer by ADENO-X RAPID titer kit (BD Biosciences), the viral vector (Ad.shGAX) was used to transduce primary human or mouse BEC for expression of the shRNA GAX specific silencer. pAd U6-GFP shRNA silencer of GFP (Ad.shGFP) was used as a control. Specific downregulation of GAX was confirmed by Western blot analysis.

GAX plasmid and adenoviral constructs. An adenoviral construct expressing the human homolog of GAX (Ad.hGAX) was from Dr. David Gorski (UMDNJ—Robert Wood Johnson Medical School, NJ)³⁰. Ad.GFP was obtained from Dr. Joseph Miano (University or Rochester). Viral titers were determined by plaque assay. Prior to use of Ad.hGAX in BEC, expression of GAX mRNA and protein in transduced cells were verified by Northern and Western blot analysis.

3-D capillary morphogenesis assay. This assay has been described in detail elsewhere²⁹. Briefly, 2×10⁶ BEC/ml from AD and age-matched controls (Table 1) or young controls were suspended within 3-D collagen matrices at 30 μL per well in the serum-free culture Medium 199 containing VEGF₁₆₅ and FGF-2 (Upstate Biotechnology, Lake Placid, N.Y.) at 40 ng/ml in 5% CO₂ at 37° C. Cultures were fixed with 3% glutaraldehyde in phosphate buffer saline and stained with toluidine blue and Hoechst 33342. The formation of the intracellular vacuoles (stage I) and tubes (stage II) were studied within 24 hr. The cells were considered to be in a vacuolar stage when ≧30% of the cell surface was occupied by vacuole(s)²⁹. Tubes were defined as elongated cells at least 15 μm in length with a lumen. Total tube length per field was measured using IMAGEPRO PLUS software. TUNEL staining was performed as described below.

Matrigel capillary tube formation assay. This was performed as previously described⁵⁰. Briefly, control human primary BEC transduced with Ad.shGAX or Ad.shGFP, primary AD BEC transduced with Ad.hGAX or Ad. GFP, and primary mouse BEC derived from Gax^{+/-} mice and littermate controls (see below) were plated on growth factor reduced Matrigel matrix (Becton Dickinson) at 2×10⁴ cells per well in 48-well plates in RPMI1640 medium containing 0.1% FBS. After four to six hours at 37° C., VEGF₁₆₅ was added to 10 ng/ml and incubation continued overnight. To quantify the tubular structures, images from four fields per well in duplicate wells were photographed at ×10 magnification with a digital camera (Spot) attached to a Nikon microscope. Total tube length per field was measured using IMAGEPRO PLUS software.

Transgenic mice. Gax^{+/-} mice²² at 2-3-month and 10-12-month of age, Tg2576 APPsw^{+/-} mice³⁵ at 18-20-month of age, and AhR^{-/-} mice³⁸ at 2-3 month of age were used. Animal studies were performed according to the National Institutes of Health guidelines using an approved institutional protocol.

Immunostaining of BEC in mouse tissue. For Gax staining on brain microvessels in Tg2576 and control mice, 14 μm frozen acetone fixed tissue sections and double immunostaining for Gax and CD31 (endothelial marker) were used. For CD31 staining, mouse CD31-specific IgG was used as a primary antibody, and Alexa Fluor 594 donkey anti-rat IgG (1:500, Molecular Probes, Inc. Eugene, Oreg.) as a secondary antibody. Ten randomly selected fields from ten sections spanning the entire cortex from four mice per group were analyzed.

Brain capillary length in mouse tissue. To determine total brain capillary length in Gax^{+/-} and AhR^{-/-} and control mice hundreds paraffin-embedded coronal sections (8 μm thickness) of each mouse were cut and 1/10 of the sections were sampled in a systematic uniform random manner for each animal.

Sections were immunostained for CD31 (PECAM-1) in order to label vessels (10 sections per mouse) and total brain capillary length determined using IMAGEPRO PLUS software.

Radioiodination of Aβ. Radioiodination of synthetic Aβ40 peptide was carried out by lactoperoxidase method as previously described¹⁴. Typically, 10 μg of Aβ40 was labeled for 18 min at room temperature with 2 mCi of Na[¹²⁵I]. After radiolabeling, the preparations were subjected to reverse-phase HPLC separation using a Vydac C4 column and a 30 min linear gradient of 25% to 40% acetonitrile in 0.059% trifluoroacetic acid to separate the monoiodinated non-oxidized form of Aβ40 (which is the tracer) from diiodinated Aβ40, nonlabeled nonoxidized Aβ40, and oxidized Aβ40 species as previously reported^{14,33}. The content of material in the peaks eluted from HPLC was determined by MALDI-TOF mass-spectrometry to ensure the purity of the radiolabeled species. For MALDI-TOF mass spectrometry Aβ peptides were labeled under identical conditions using Na[¹²⁷] instead of the radioactive nuclide. The specific activity was in the range of 45 to 65 μCi/μg of peptide. For clearance studies, preparations were usually used within 24 hr of labeling that was ≧99% TCA-precipitable. If used within 72 hr of labeling, the radiolabeled peptides were stabilized in ethanol as a quenching agent. Prior to each in vitro study or infusion into animals, the tracer was purified by HPLC. The HPLC/SDS-PAGE analysis was used to confirm the monomeric state of infused radiolabeled Aβ40.

Brain clearance studies in mice. CNS clearance of ¹²⁵I-labeled Aβ40 was determined simultaneously with ¹⁴C-inulin (reference marker) in Gax^{+/-} mice and littermate controls 8-10 weeks old, using a procedure as described^{13,14}. Calculation of clearance parameters was performed as reported¹⁴. Briefly, a stainless steel guide cannula was implanted stereotaxically into the right caudate-putamen of anesthetized mice (0.5 mg/kg ketamine and 5 mg/kg xylazine I.P.). Coordinates for tip of the cannula were 0.9 mm anterior and 1.9 mm lateral to the bregma and 2.9 mm below the surface of the brain. Animals were allowed to recover after surgery prior to radiotracer studies. The experiments were performed before substantial chronic processes have occurred, as assessed by histological analysis of tissue, i.e., negative staining for astrocytes (glial fibrillar acidic protein) and activated microglia (anti-phosphotyrosine), but allowing time for BBB repair to large molecules, typically four to six hours after the cannula insertion as reported⁹. Tracer fluid (0.5 μL) containing [¹²⁵I]-Aβ40 and ¹⁴C-inulin was injected into brain ISF over 5 min via an ultra micropump with a MICRO4 controller (World Precision Instruments, Sarasota, Fla.). Brain and blood were sampled 30 min after tracers injection and prepared for radioactivity analysis as described¹⁴. Gamma counting was performed using Wallac VIZARD gamma counter (Perkin Elmer, Meriden, Conn.) and beta-counting using TRI-CARB 2100 liquid scintillation counter (Perkin Elmer, Conn.). Previous studies with ¹²⁵I-labeled Aβ demonstrated an excellent correlation between TCA and HPLC methods. The intactness of ¹²⁵I-labeled Aβ40 injected into the brain ISF was >99% by TCA/HPLC analysis. The Aβ40 standards eluted at 29.8 min. For SDS-PAGE analysis, TCA precipitated samples were resuspended in 1% SDS, vortexed and incubated at 55° C. for 5 min, then neutralized, boiled for 3 min, homogenized and analyzed by electrophoresis in 10% Tris-tricine gels followed by fluorography. Methodological details were as reported^{13,14}.

The percentage of radioactivity remaining in the brain after microinjection was determined as % recovery in brain=100×(N_{b}/Nᵢ) (1), where, N_{b} is the radioactivity remaining in the brain at the end of the experiment and Nᵢ is the radioactivity injected into the brain ISF, i.e., the d.p.m. for ¹⁴C-inulin and the c.p.m. for TCA-precipitable ¹²⁵I-radioactivity (intact Aβ). The percentage of Aβ cleared through the BBB was calculated using the formula [(1−N_{b(Aβ)}/N_{i(Aβ)})−(1−N_{b(inulin)}/Nᵢ₍ᵢₙᵤₗᵢₙ₎)]×100, using a standard time of 30 min (2).

Cerebral blood flow in mice. The CBF was studied with ¹⁴C-iodoantipyrine (¹⁴C-IAP; Amersham)³³. Gax^{+/-} and control mice were infused with 0.15 μCi of ¹⁴C-IAP and after 30 s the heads immediately immersed in liquid nitrogen. The frozen brains were sectioned at 20 μm, mounted on slides, and representative sections exposed to HYPERFILM βMAX autoradiographic film (Amersham) along with ¹⁴C standards. After a three day exposure, the film was developed and the resulting images analyzed by quantitative autoradiography on an MCID image analyzer (Imaging Research) to determine levels of ¹⁴C-IAP. The CBF was calculated using the basic equation CBF=−λ ln (1−C_{IN (T)}/λ C_{PL})/T, where C_{IN (T)} is activity in unit mass of brain at time T, C_{PL} is the integrated concentration of ¹⁴C-IAP in arterial inflow, and λ is the distribution ratio of ¹⁴C-IAP between brain and plasma at steady state, which equals 0.8.

Hypoxia model. Male Gax^{+/+} and Gax^{+/-} mice 2-3-month old were exposed to hypoxia as described³⁴. A normobaric chamber with 10% oxygen was used on the first day, 9% on the second day, and then followed by 8% of oxygen for up to three weeks. Brains were analyzed after three weeks for capillary density and at four days for the levels of VEGF, GAX, AFX1, and Bcl_{XL}.

Vascular contractility assay. The thoracic aorta, free from connective tissues, was isolated and removed from anesthetized (50 mg/kg ketamine and 5 mg/kg xylazine i.p.) Gax^{+/+} and Gax^{+/-} mice. Three mm sections were used to determine contraction and relaxation using a 10 ml Radnoti organ bath system and Grass myograph (Grass-Telefactor Instruments, Warwick, R.I.). Tissue was bathe in Krebs solution, gassed continuously with 95% O₂ and 5% CO₂ at pH 7.4 and at 37±0.5° C. The resting tension was maintained at 0.5 g. Cumulative dose-response curves for contraction to phenylephrine and relaxation to acetylcholine following pre-contraction with 0.25×10⁻⁶ mol/l phenylephrine were determined.

Mouse BEC cultures. Primary cultures of mouse microvascular BEC were established as described⁴⁹. Briefly, six to ten mice were used each time. Cerebral cortices were cut into small pieces and homogenized in MCDB131 medium containing 2% FBS, 100 U/ml penicillin and 100 μg/ml streptomycin. The microvessels were isolated from the homogenate by fractionation on a 15% dextran gradient, and then digested with 0.1% collagenase/dispase (Boehringer Mannheim, Indianapolis, Ind.) in MCDB131 medium containing 2% FBS for six hours at 37° C. After centrifugation on a 45% PERCOLL gradient, the digested micro-vessels and dissociated endothelial cells in the top layer were cultured in MCDB 131 medium supplemented with 30 μg/ml ECGS (Sigma), 10% FBS, 15 U/ml heparin, 325 μg/ml glutathione, 1 μl/ml 2-mercaptoethanol, 100 U/ml penicillin and 100 μg/ml streptomycin (all from Sigma) on collagen 1-coated (Roche Diagnostics, Mannheim, Germany) plastic ware. BEC were further purified using rat anti-mouse CD31 antibodies (BD Pharmagen, Lexington, Ky.) and Dynabeads M-450 sheep anti-Rat IgG (Dynal Biotech, Oslo, Norway) magnetic beads.

Metabolic labeling of BEC. Human BEC (4×10⁵) were pulsed for one hour at 37° C. with 400 μCi of [³⁵S]-methionine (>1000 Ci/mmol; Perkin Elmer, Boston, Mass.) in methionine-free Dulbecco modified Eagle medium (Gibco BRL, New York, N.Y.) as described¹⁴. Human BEC (4×10⁵) were pulsed for one hour at 37° C. with 400 μCi of [³⁵S]-methionine (>1000 Ci/mmol; Perkin Elmer, Boston, Mass.) in methionine-free Dulbecco modified Eagle medium (Gibco BRL, New York, N.Y.) as described⁹. Cells were chased at indicated times within 48 hours. Cell lysates were immunoprecipitated with LRP-515 kDa α-chain specific IgG (8G1) on SDS-PAGE. The intensity of signal was quantified in pixels using the Storm 860 PHOSPHOIMAGER (Amersham Biosciences, Piscataway, N.J.).

Aβ treatment. Human BEC were treated for 24 hr with different concentrations of Aβ42 ranging from 0.1 to 1,000 nM. Oligomeric and aggregated forms of Aβ42 were prepared as described⁵⁰.

Western blot analysis. Cell lysates were prepared for Western blot analysis as described¹⁴. Gax, rabbit polyclonal antibody to C-terminal region of the rat Gax protein that cross reacts with human GAX homeoprotein (amino acids SDHSS EHAHL, SEQ ID NO:2), 1:500 (7 mg/ml)⁴⁹; polyclonal rabbit antibody to human AFX1 (1:1000, 0.1 mg/ml; Sigma, St. Louis, Mo.); polyclonal rabbit antibody to human Bcl-X_{L} (1:200, 0.2 mg/ml; Santa Cruz Biotechnology, Santa Cruz, Calif.); monoclonal mouse antibody to human ankyrin G (1:100, 0.2 mg/ml; Santa Cruz Biotechnology); polyclonal goat antibody to human plectin (1:100, 0.2 mg/ml; Santa Cruz Biotechnology); monoclonal mouse antibody to C-terminal domain of human LRP-1 β-chain which cross reacts with mouse LRP-1 (5A6, 1:350, 5 μg/ml; EMD Biosciences, San Diego, Calif.); monoclonal mouse antibody to human LRP-1 α-chain (8G1, 1:240, 5 μg/ml; EMD Biosciences) and β-actin, goat anti-human polyclonal, 1:2,500 (0.2 mg/ml, Santa Cruz Biotechnology); polyclonal goat antibody to human MEF2 (1:500, 0.2 mg/ml; Santa Cruz Biotechnology); polyclonal rabbit antibody to human VEGF (1:100, 0.2 mg/ml; Santa Cruz Biotechnology); polyclonal rabbit antibody to human Bax (1:1000, Cell Signaling, Beverly, Mass.); mouse monoclonal antibody to hemaglutinin (HA) (1:200, 0.2 mg/ml; Santa Cruz Biotechnology); mouse monoclonal antibody to human RAP (1:500, 25 μg/ml; EMD Biosciences); monoclonal mouse antibody to human transferrin receptor (1:500, 1 μg/ml; Zymed Laboratories, South San Francisco, Calif.); and polyclonal rabbit antibody to rat Tinur which crossreacts with human TINUR (1:200, 0.2 mg/ml; Santa Cruz Biotechnology) were used.

TUNEL assay. Staining with APOPTAG kit (TUNEL) was performed according to the manufacturer's instructions (Intergen, Purchase, N.Y.).

Statistical analysis. ANOVA was used to determine statistically significant differences. P<0.05 was considered as statistically significant.

TABLE 1

| | | | | | | | | | |
|---|---|---|---|---|---|---|---|---|---|
| Patient | | | PMI | Cause of | Vascular | | | | |
| Number | Age | Gender | (hr) | Death | Risk Factors | Angiopathy | Braak | CERAD | CDR |

Alzheimer's Disease Patients

| | | | | | | | | | |
|---|---|---|---|---|---|---|---|---|---|
| 01 | 66 | M | 5.0 | Cardiac | Atherosclerosis | + | V-VI | Frequent | 3 |
| | | | | Arrest | | | | | |
| 03 | 86 | F | 3.0 | Respiratory | Atherosclerosis | + | V-VI | Frequent | 3 |
| | | | | Failure | | | | | |
| 15 | 67 | F | 4.2 | Respiratory | Atherosclerosis | + | V-VI | Moderate | 4 |
| | | | | Failure | | | | | |
| 20 | 70 | M | 5.0 | Pneumonia | None | + | V-VI | Moderate | 4 |
| 36 | 80 | M | 2.3 | Cardiac | Hypertension | + | V-VI | Moderate | 4 |
| | | | | Arrest | | | | | |
| 45 | 66 | F | 4.0 | Pneumonia | None | + | V-VI | Frequent | 4 |

Age-Matched Neurologically Normal Subjects (Controls)

| | | | | | | | | | |
|---|---|---|---|---|---|---|---|---|---|
| 13 | 92 | F | 6.9 | Cardiac | Atherosclerosis | + | 0-I | Moderate | 0.5 |
| | | | | Arrest | | | | | |
| 14 | 88 | M | 1.4 | Respiratory | Atherosclerosis | + | 0-I | Sparse | 0 |
| | | | | Arrest | | | | | |
| 16 | 64 | M | 4.5 | Cardiac | Hypertension | − | 0-I | Negative | 0 |
| | | | | Arrest | Atherosclerosis | | | | |
| 17 | 59 | F | 4.5 | Stroke | None | − | 0 | Negative | 0 |
| 38 | 58 | F | 5.5 | Pulmonary | None | − | 0 | Negative | 0 |
| | | | | Embolisum | | | | | |
| 39 | 72 | M | 4.3 | Cardiac | Atherosclerosis | − | 0-I | Sparse | 0 |
| | | | | Arrest | Myocardial | | | | |
| | | | | | Infarct | | | | |

PMI, post-mortem interval; Angiopathy, cerebral amyloid angiopathy; CERAD, Consortium to Establish Registry for Alzheimer's Disease; CDR, Clinical Dementia Rating score.

TABLE 2

| | | | AD vs. age- | | |
|---|---|---|---|---|---|
| | | Accession | matched | | Old vs. |
| Function | Gene Name | Number | Fold Δ | P | Young |
| Cell | Mesenchyme homeobox 2/growth | AI743406 | −2.0 | 0.003 | NS |
| Differentiation | arrest-specific homeobox; | | | | |
| | MEOX2/Gax | | | | |
| | TINUR = NGFI-B/nur77 beta-type | S77154 | −7.0 | 0.05 | NS |
| | transcription factor; nuclear receptor | | | | |
| | subfamily 4, group A, member 2 | | | | |
| | (NR4A2) | | | | |
| Signal | Phosphodiesterase 1A, calmodulin- | U40370 | −3.0 | 0.01 | NS |
| transduction | dependent; PDE1A | | | | |
| | Plectin 1; PLEC1 | Z54367 | −3.0 | 0.03 | NS |
| | RAS (RAD and GEM)-like GTP- | AF084465 | −2.6 | 0.03 | NS |
| | binding 1; REM1 | | | | |
| | CD2 antigen (cytoplasmic tail) binding | AF104222 | −2.5 | 0.04 | NS |
| | protein 2; CD2BP2 | | | | |
| | Ankyrin G (ANK3) | U13616 | −2.2 | 0.03 | NS |
| | Phosphodiesterase 1B, calmodulin- | U86078 | −2.2 | 0.02 | NS |
| | dependent; PDE1B | | | | |
| Protein | Ribosomal protein L37a; RPL37A | L06499 | −3.8 | 0.004 | NS |
| Turnover | Eukaryotic translation initiation factor | L19161 | −2.5 | 0.007 | NS |
| | 2, subunit 3 gamma; EIF2S3 | | | | |
| | Heat shock 70 kDa protein 2; HSPA2 | L26336 | −2.5 | 0.03 | NS |
| Matrix | A disintegrin-like and metalloprotease | AJ003125 | −2.8 | 0.05 | NS |
| | (reprolysin type) with thrombospondin | | | | |
| | type 1 motif, 2; ADAMTS2- | | | | |
| | Adipose most abundant gene | AI381790; | −2.8 | 0.04 | NS |
| | transcript 2; APM2 | D45370 | | | |
| | Elastin, ELN | X52896 | −2.4 | 0.04 | NS |
| Cell Cycle | Cell division protein kinase 10; CDK10 | X78342 | −2.5 | 0.01 | NS |
| | (Serine/threonine-protein kinase | | | | |
| | PISSLRE) | | | | |
| | Interferon induced transmembrane | J04164 | −2.2 | 0.04 | NS |
| | protein 1 (9-27); IFITM1 | | | | |
| | p8 protein (candidate of metastasis 1); | AI557295 | −2.1 | 0.05 | NS |
| | p8 | | | | |
| | Myeloid translocation gene-related | AB010419 | 2.1 | 0.01 | NS |
| | protein 2 isoform; MTG16a | | | | |
| | Histone 2, H2ac; HIST2H2AC | Z80776 | 2.2 | 0.04 | NS |
| Apoptosis | V-maf musculoaponeurotic | AF055376 | −3.1 | 0.005 | NS |
| | fibrosarcoma oncogene homolog | | | | |
| | (avian), (Protooncogene C-maf): MAF | | | | |
| | Forkhead domain transcription factor; | Y11284 | 2.0 | 0.05 | NS |
| | AFX1 | | | | |
| Metabolism | Fatty acid desaturase 2; FADS2 | AL050118 | −2.2 | 0.05 | NS |
| | Cytochrome P-450, family 11, | X55764 | 2.5 | 0.005 | NS |
| | subfamily B; (CYP11B) | | | | |
| | Phosphoserine-phosphatase; PSPH | AJ001612 | 5.3 | 0.04 | +3.2 |
| | | | | | P < 0.05 |
| | Transglutaminase 2; TGM2 | M55153 | 5.8 | 0.00 | NS |
| | Apurinic/apyrimidinic endonuclease 2, | AJ011311 | 2.2 | 0.002 | NS |
| | APEX2 | | | | |
| | Ectonucleoside triphosphate | AJ133133 | 2.1 | 0.01 | NS |
| | diphosphohydrolase 1 (CD39); | | | | |
| | ENTPD1 | | | | |
| Immunity | Defensin, beta 4; DEFB4 | AF071216 | −2.0 | 0.02 | NS |
| | FK506-binding protein 1A, FKBP1A | NM_054014 | 2.4 | 0.008 | NS |
| | Six transmembrane epithelial antigen | AC005053 | 2.2 | 0.05 | NS |
| | of the prostate (STEAP) | | | | |
| Growth | Glial growth factor 2, neuregulin 1, | L12260 | 2.1 | 0.01 | NS |
| Factors | NRG1 | | | | |
| Cell Surface | Brain-specific angiogenesis inhibitor 2; | AB005298 | 2.3 | 0.03 | NS |
| Receptors | BAI2 | | | | |
| | Protocadherin 9; PCDH9 | AF169692 | 2.3 | 0.05 | NS |
| Protein | Ankyrin repeat and BTB domain | AL050374 | 2.3 | 0.05 | NS |
| Binding | containing 2; ABTB2 | | | | |

The analysis included six AD patients, six age-matched controls, and five young controls. Details about patients and controls are given in Supplementary Table 1. Statistical analysis was performed using Bayesian T-test¹⁸. See Methods for details.

### REFERENCES

1. Alzheimer (1907) Uber eine eigenartig Erkrankung der Hirnrinde. Allg. Z. Psychiatrie Psych. Ger. Med. 64, 146-148.
2. Zlokovic (2005) Neurovascular mechanisms of Alzheimer's neurodegeneration. Trends Neurosci. 28, 202-208.
3. de la Torre (2004) Alzheimer's disease is a vasocognopathy: A new term to describe its nature. Neurol Res. 26, 517-524.
4. Gorelick (2004) Risk factors for vascular dementia and Alzheimer's disease. Stroke 35, 2620-2622.
5. Casserly & Topol (2004) Convergence of atherosclerosis and Alzheimer's disease: inflammation, cholesterol, and misfolded proteins. Lancet 363, 1139-1146.
6. Roher et al. Atherosclerosis of cerebral arteries in Alzheimer's disease. Stroke 35, 2623-2627 (2004).
7. Greenberg et al. Amyloid angiopathy-related vascular cognitive impairment. Stroke 35, 2616-2619 (2004).
8. Bailey et al. (2004) The nature and effects of cortical microvascular pathology in aging and Alzheimer's disease. Neurol Res. 26, 573-578.
9. Farkas & Luiten (2001) Cerebral microvascular pathology in aging and Alzheimer's disease. Prog. Neurobiol. 64, 575-611.
10. Paris et al. (2004) Impaired angiogenesis in a transgenic mouse model of cerebral amyloidosis. Neurosci. Lett. 366, 80-85.
11. Paris et al. (2004) Inhibition of angiogenesis by Aβ peptides. Angiogenesis 7, 75-78.
12. Carmeliet & Jain (2003) Angiogenesis in cancer and other diseases. Nat. Med. 9, 653-660.
13. Shibata et al. (2000) Clearance of Alzheimers amyloid-β 1-40 peptide from brain by LDL receptor related protein-1 at the blood-brain barrier. J. Clin. Invest. 106, 1489-1499.
14. Deane et al. (2004) LRP/amyloid β-peptide interaction mediates differential brain efflux of Aβ isoforms. Neuron 43, 333-344.
15. Tanzi et al. (2004) Clearance of Alzheimer's Aβ peptides: the many roads to perdition. Neuron 43, 605-608.
16. Iadecola (2004) Neurovascular regulation in the normal brain and in Alzheimer's disease. Nat. Neurosci. Rev. 5, 347-360.
17. Iadecola et al. (1999) SOD1 rescues cerebral endothelial dysfunction in mice overexpressing amyloid precursor protein. Nat. Neurosci. 2, 157-161.
18. Gorski et al. (1993) Molecular cloning of a diverged homeobox gene that is rapidly downregulated during the Go/G1 transition in vascular smooth muscle cells. Mol. Cell. Biol. 13, 3722-3733.
19. Gorski & Walsh (2003) Control of vascular cell differentiation by homeobox transcription factors. Trends Cardivasc. Med. 13, 213-220.
20. Tang et al. (2002) The Forkhead transcription factor AFX activates apoptosis by induction of the BCL-6 transcriptional repressor. J. Biol. Chem. 277, 14255-14265.
21. Bu & Marzolo (2000) Role of RAP in the biogenesis of lipoprotein receptors. Trends Cardiovascular Med. 10, 148-155.
22. Mankoo et al. (1999) Mox2 is a component of the genetic hierarchy controlling limb muscle development. Nature 400, 69-73.
23. Braak & Braak (1991) Neuropathological staging of Alzheimer-related changes. Acta Neuropathol. 82, 239-259.
24. Hyman & Trojanowski (1997) Consensus recommendations for the postmortem diagnosis of Alzheimer disease from the National Institute on Aging and the Regan Institute Working Group on diagnostic criteria for the neuropathological assessment of Alzheimer disease. J. Neuropathol. Exp. Neurol. 56, 1095-1097.
25. Baldi & Long (2001) A Bayesian framework for the analysis of microarray expression data: regularized t-test and statistical inferences of gene changes. Bioinformatics 17, 509-519.
26. Liu et al. (2003) Vascular endothelial growth factor regulated gene expression in endothelial cells: KDR-mediated induction of Egr3 and the related nuclear receptors Nur77, Nurr1, and Nor1. Arterioscier. Thromb. Vasc. Biol. 23, 2002-2007.
27. Citron et al. (2002) Protein crosslinking, tissue transglutaminase, alternative splicing and neurodegeneration. Neurochem. Intl. 40, 69-78.
28. Kozak (1999) Initiation of translation in prokaryotes and eukaryotes. Gene 234, 187-208.
29. Davis et al. (2001) Matrix metalloproteinase-1 and -9 activation by plasmin regulates a novel endothelial cell-mediated mechanism of collagen gel contraction and capillary tube regression in three-dimensional collagen matrices. J. Cell Sci. 114, 917-930.
30. Gorski & Leal (2003) Inhibition of endothelial cell activation by the homeobox gene Gax. J. Surg. Res. 111, 91-99.
31. Patel et al. (2005) The homeobox gene Gax inhibits angiogenesis through inhibition of nuclear factor-kB-dependent endothelial cell gene expression. Cancer Res. 65, 1414-1424.
32. Perlman et al. (1998) Bax-mediated cell death by the Gax homeoprotein requires mitogen activation but is independent of cell cycle activity. EMBO J. 17, 3576-3586.
33. LaRue et al. (2004) Method for measurement of the blood-brain barrier permeability in the perfused mouse brain: application to amyloid β-peptide in wild type and Alzheimer's Tg2576 mice. J. Neurosci. Methods 138, 233-242.
34. LaManna et al. (2004) Structural and functional adaptation to hypoxia in the rat brain. J. Exp. Biol. 207, 3163-3169.
35. Kawarabayashi et al. (2001) Age dependent changes in brain, CSF, and plasma amyloid-β in the Tg2576 transgenic mouse model of Alzheimer's disease. J. Neurosci. 21, 372-381.
36. Fryer et al. (2003) Apolipoprotein E markedly facilitates age-dependent cerebral amyloid angiopathy an spontaneous hemorrhage in amyloid precursor protein transgenic mice. J. Neurosci. 23, 7889-7896.
37. Herz & Marschang (2003) Coaxing the LDL receptor family into the blood. Cell 112, 289-292.
38. Lahvis et al. (2000) Portosystemic shunting and persistent fetal vascular structures in aryl hydrocarbon receptor-deficient mice. Proc. Natl. Acad. Sci. USA 97, 10442-10447.
39. Li et al. (2001) Differential functions of members of the low density lipoprotein receptor family suggested by their distinct endoytosis rates. J. Biol. Chem. 276, 18000-18006.
40. Andres et al. (1995) Regulation of Gax homeobox gene transcription by a combination of positive factors including myocyte-specific enhancer factor 2. Mol. Cell Biol. 15, 4272-4281.
41. Saito et al. (2005) Angiotensin II suppresses growth arrest specific homeobox (Gax) expression via redox-sensitive mitogen-activated protein kinase (MAPK). Reg. Peptides 127, 159-167.
42. Atwood et al. (2003) Amyloid-β: a chameleon walking in two worlds: a review of the trophic and toxic properties of amyloid-beta. Brain Res. Rev. 43, 1-16.
43. Hermann et al. (2001) Relationship between metabolic dysfunctions, gene responses and delayed cell death after mild focal cerebral ischemia in mice. Neuroscience 104, 947-955.
44. Iscove et al. (2002) Representation is faithfully preserved in global cDNA amplified exponentially from sub-picogram quantities of mRNA. Nat. Biotechnol. 20, 940-943.
45. Holland et al. (1991) Detection of specific polymerase chain reaction product by utilizing the 5′,3′ exonuclease activity of Thermus aquaticus DNA polymerase. Proc. Natl. Acad. Sci. USA 88, 7276-7280.
46. Mackic et al. (1998) Human blood-brain barrier receptors for Alzheimer's amyloid-β. J. Clin. Invest. 102, 734-743.
47. Russell et al. (2000) Update on adenovirus and its vectors. J. Gen. Virol. 81, 2573-2604.
48. Wu et al. (2003) A simple method for isolation and characterization of mouse brain microvascular endothelial cells. J. Neurosci. Methods 130, 53-63.
49. Witzenbichler et al. (1999) Regulation of smooth muscle cell migration and integrin expression by the Gax transcription factor. J. Clin. Invest. 104, 1469-1480.
50. Kayed et al. (2003) Common structure of soluble amyloid oligomers implies common mechanism of pathogenesis. Science 300, 486-489.

Patents, patent applications, books, and other publications cited herein are incorporated by reference in their entirety.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transition “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A treatment method which at least promotes angiogenesis, suppresses apoptosis, or increases low density lipoprotein receptor-related protein 1 (LRP-1) mediated clearance of amyloid p peptide (Aβ), said method comprising:
(a) inserting a nucleic acid comprised of a GAX gene into one or more neurovascular cells and
(b) expressing GAX in said neurovascular cells from said nucleic acid which is effective to promote angiogenesis, to suppress apoptosis, or to increase LRP-1 mediated clearance of Aβ.

2. The method of claim 1, wherein GAX expression in vascular endothelium of a subject's blood-brain barrier before said treatment is reduced to an amount indicative of Alzheimer's disease pathogenesis.

3. The method of claim 1, wherein neurovascular dysfunction in a subject's blood-brain barrier is corrected by said treatment.

4. The method of claim 2, wherein the subject is a human.

5. The method of claim 1, wherein GAX expression decreases expression of AFX1 forkhead transcription factor in one or more neurovascular cells.

6. The method of claim 1, wherein GAX expression increases expression of receptor associated protein (RAP) in one or more neurovascular cells.

7. The method of claim 1 further comprising (c) confirmation that GAX expression is increased in one or more neurovascular cells by said treatment.

8. The method of claim 1, wherein human GAX is expressed by said nucleic acid.

9. The method of claim 1, wherein nucleic acid insertion or GAX expression or both are directed at least to one or more neurovascular cells.

10. The method of claim 1, wherein said nucleic acid is further comprised of a drug-inducible or endothelial cell-specific or virus regulatory region directing GAX transcription.

11. The method of claim 10, wherein said regulatory region is from a gene selected from the group consisting of angiopoietin receptors (Tie-1 and Tie-2), endoglin, endothelin-1 (ET1), intercellular adhesion molecule (ICAM-2), vascular endothelial growth factor receptors (FLT-1 and FLK-1), and vascular endothelial growth factor (VEGF).

12. The method of claim 1, wherein said nucleic acid is replicated in a virus vector at least in one or more neurovascular cells.

13. The method of claim 12, wherein said viral vector is contained in a carrier which delivers its contents at least into one or more neurovascular cells.

14. The method of claim 13, wherein said carrier is a virus particle or a liposome.

15. The method of claim 10, wherein said virus is selected from the group consisting of adenovirus, adeno-associated virus, cytomegalovirus, fowlpox virus, herpes simplex virus, lentivirus, Moloney leukemia virus, mouse mammary tumor virus, Rous sarcoma virus, SV40 virus, and vaccinia virus.

16. The method of claim 12, wherein said virus is selected from the group consisting of adenovirus, adeno-associated virus, cytomegalovirus, fowlpox virus, herpes simplex virus, lentivirus, Moloney leukemia virus, mouse mammary tumor virus, Rous sarcoma virus, SV40 virus, and vaccinia virus.

17. The method of claim 14, wherein said virus is selected from the group consisting of adenovirus, adeno-associated virus, cytomegalovirus, fowlpox virus, herpes simplex virus, lentivirus, Moloney leukemia virus, mouse mammary tumor virus, Rous sarcoma virus, SV40 virus, and vaccinia virus.

18-20. (canceled)


SEQUENCE LISTING (2 sequences)

SEQ ID NO: 1 - DNA, 21 bp, Artificial Sequence
  Insert for silencing double-stranded oligonucleotide
ggaaggaaattacaagtcaga

SEQ ID NO: 2 - protein, 10 aa, Artificial Sequence
  Oligopeptide antigen for human GAX homeoprotein
SDHSSEHAHL